(19) United States
(12) Patent Application Publication
(10) Pub. No.: US 2004/0040051 A1
(43) Pub. Date: Feb. 26, 2004

(54) Generation of non-human mammals by nuclear cloning

(75) Inventors: William M. Rideout III, Cambridge, MA (US); Teruhiko Wakayama, Minatoshimachuou (JP); Kevin C. Eggan, Cambridge, MA (US); Ryuzo Yanagimachi, Honolulu, HI (US); Hidenori Akutsu, Baltimore, MD (US); Rudolf Jaenisch, Brookline, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignees: Whitehead Institute for Biomedical Research, Cambridge, MA; The University of Hawaii, Honolulu, HI

(21) Appl. No.: 10/426,247

(22) Filed: Apr. 29, 2003

Related U.S. Application Data
(63) Continuation of application No. 10/264,347, filed on Oct. 3, 2002, now abandoned.
Application No. 10/264,347 is a continuation of application No. 10/051,622, filed on Jan. 18, 2002, now abandoned.
(60) Provisional application No. 60/262,957, filed on Jan. 19, 2001.

(51) Int. Cl. A01K 67/027

(52) U.S. Cl. 800/18; 800/21

(57) ABSTRACT

A method of producing a non-human mammalian embryo, such as a mouse embryo, by nuclear cloning, in which the nucleus from a non-human mammalian embryonic stem (ES) cell (e.g., a non-human mammalian F1 ES cell), such as the nucleus of a mouse F1 ES cell, is introduced into an enucleated non-human mammalian oocyte, such as an enucleated mouse oocyte; embryos produced by the method; a method of producing mice from the resulting embryos and the mice produced thereby.

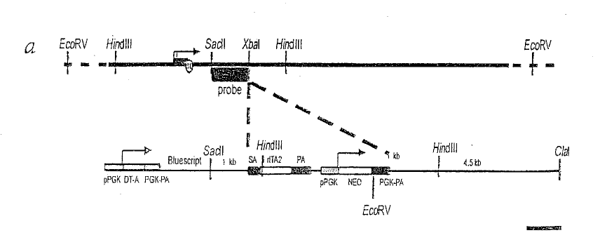

### RELATED APPLICATIONS

This application is a continuation of U.S. application Ser. No. 10/264,347, filed Oct. 3, 2002, which is a continuation of U.S. application Ser. No. 10/051,622, filed Jan. 18, 2002, which claims the benefit of U.S. Provisional Application No. 60/262,957, filed on Jan. 19, 2001. The entire teachings of the above applications are incorporated herein by reference.

### GOVERNMENT SUPPORT

Work described herein was supported, in whole or in part, by Grant No. 5-R35-CA44339 from the National Institutes of Health. The United States government has certain rights in the invention.

### BACKGROUND OF THE INVENTION

Mammalian cloning has recently been developed (Baguisi, A. et al., Nat. Biotechnol., 17:456-461 (1999); Kato, Y. et al., Science, 282:2095-2098 (1998); Wells, D. N. et al., Biol. Reprod. 60:996-1005 (1999); Wilmut, I. et al., Nature, 385:810-813 (1997); and Wakayama, T. et al., Nature, 394:369-374 (1998)), but a major problem has been the low frequency of viable clones as most clones die during gestation or soon after birth. Parameters which affect cloning efficiency may include genetic background, passage number, cell cycle stage of the donor cell (Campbell, K. H. et al., Rev. Reprod., 1:40-46 (1996)), loss of imprints, accumulated genetic damage of the donor cells, or the ability of the oocyte to epigenetically reprogram the donor cell nucleus. Cloning in mice (Wakayama, T. et al., Nature, 394:369-374 (1998); Wakayama, T. et al., Nature Genet., 22:127-128 (1999); and Wakayama, T. et al., Proc. Natl. Acad. Sci. USA, 96:14984-14989 (1999)), the best mammalian model organism, has until recently been limited to freshly isolated or primary cultures of somatic cells, which limits study of the parameters that affect cloning efficiency.

### SUMMARY OF THE INVENTION

The present invention relates to Applicants' discovery that established and targeted embryonic stem (ES) cells or cell lines can generate cloned embryos (e.g., cloned non-human mammalian embryos) and animals (e.g., non-human mammals, such as mice), thus making possible the study of parameters important for cloning.

The present invention relates to a method of producing cloned non-human mammalian embryos, which can be mutant or non-mutant embryos. The method comprises transferring a nucleus from a non-human mammalian ES cell (e.g., F1 ES cell) into an enucleated non-human mammalian oocyte to produce an enucleated non-human mammalian oocyte having the nucleus from the non-human mammalian ES cell incorporated therein. The resulting enucleated non-human mammalian oocyte having the nucleus from the non-human mammalian ES cell incorporated therein is activated, thereby producing an activated oocyte. The activated oocyte is cultured under conditions appropriate for blastocyst (embryo) development, thereby producing a cloned non-human mammalian embryo. In a particular embodiment, the non-human mammalian ES cell is a mouse cell and the enucleated non-human mammalian oocyte is a mouse cell. In a specific embodiment, the non-human mammalian ES cell is a 129SvJae×C57BL/6 ES cell. In an additional embodiment, the enucleated non-human mammalian oocyte is a B6D2F1 enucleated oocyte. The ES cell can be a non-targeted (unmodified) ES cell or a targeted ES cell, such as a targeted ES cell that comprises a gene introduced into a site in genomic DNA of the cell (e.g., by homologous recombination). In one embodiment, the site in genomic DNA of the cell is a ROSA locus, such as the ROSA26 locus.

The present invention also relates to a method of producing cloned non-human mammals, which can be mutant non-human mammals or non-mutant non-human mammals, such as mice, that does not require production of chimera or chimeric offspring (offspring that consist of cells that are derived from more than one zygote). The method comprises transferring into an appropriate foster mother an embryo produced by transferring a nucleus from a non-human mammalian ES cell (e.g., F1 ES cell) into an enucleated non-human mammalian oocyte, thereby producing an enucleated non-human mammalian oocyte having incorporated therein the nucleus from the non-human mammalian ES cell; activating the enucleated non-human mammalian oocyte having incorporated therein the nucleus from the non-human mammalian ES cell, thereby producing an activated oocyte; and culturing the activated oocyte under conditions appropriate for blastocyst (embryo) development, a result of which is production of a cloned non-human mammalian embryo. The foster mother is maintained under conditions appropriate for production of offspring, and a non-human mammal is produced. A particular embodiment of this invention is a method of producing a cloned mouse, comprising (a) transferring into a pseudopregnant female mouse an embryo produced by the method described above, wherein the non-human mammalian ES cell (e.g., F1 ES cell) is a mouse cell and the enucleated non-human mammalian oocyte is a mouse cell and (b) maintaining the female mouse under conditions appropriate for production of offspring, thereby producing a cloned mouse. Another particular embodiment of this invention is a method of producing a cloned mouse, comprising transferring an embryo produced using a non-human mammalian F1 ES cell that is a 129vJae×C57BL/6 ES cell and an enucleated mouse oocyte such as a B6D2F1 enucleated oocyte.

Multiple mutations or alterations can be included in the ES cell or cell line before producing an embryo or animal from the ES cell or cell line. As is evident from the work described herein, mutant or targeted offspring, particularly mice, that are entirely derived from ES cells or ES cell lines and survive postnatally have been produced without the need to produce chimeric intermediates. Mutations introduced into ES cells or cell lines can be non-random or targeted alterations or can be random or non-targeted alterations. The products of either approach are referred to herein as mutant. In those embodiments in which mutations are non-random or targeted, the resulting products can also be referred to as targeted (e.g., targeted ES cells, targeted ES cell lines, targeted non-human mutant mammals, such as targeted mutant mice). Alterations can be of a variety of types, including deletion, addition, substitution, or modification of all or a portion of DNA (e.g., a gene, regulatory element) in the ES cells. These alterations include addition of a gene or gene portion not normally present in the ES cells or ES cell lines. Non-mutant mice that are derived entirely from ES cells or ES cell lines and survive postnatally can also be produced using the method described.

The present invention also relates to cloned non-human mammals (mutants and non-mutants), such as cloned mice, produced by the nuclear cloning methods of the invention; cells obtained from the cloned non-human mammals and cell lines produced from these cells. The invention further relates to cloned non-human mammalian embryos (mutant and non-mutant) produced by the nuclear cloning methods of the invention.

In particular embodiments, cloned mutant non-human mammals (e.g., cloned mutant mice) are produced to mimic or serve as a model for a condition (e.g., a neurological, muscular or respiratory condition, cancer, viral infection, arthritis, etc) that occurs in another species, such as in humans. They are used to identify new drugs that have a therapeutic or preventive effect on the condition or assess the ability of known drugs to act as therapeutics or preventatives. Thus, the present invention encompasses methods in which the cloned mutant non-human mammals (particularly cloned mutant mice) are used, such as in a method of screening to identify a new drug that inhibits the occurrence of (prevents the onset, reduces the extent or severity of) or reverses a condition caused by or associated with the genetic alteration(s) and a method of screening known drugs for those that inhibit onset of or reverse such conditions. Drugs identified by methods in which the cloned mutant mammals of the present invention are used are also the subject of this invention. These include drugs that inhibit onset of a condition (prevent the onset or reduce the extent to which the condition is established or severity of the condition), referred to as preventatives or prophylactic drugs and drugs that reverse (partially or completely) or reduce the extent or duration of the condition once it has occurred.

### BRIEF DESCRIPTION OF THE DRAWING

The FIGURE is a schematic of the ROSA26 genomic locus (top) and the targeting vector. Restriction sites, location of the probe used in genotyping and features of the targeting vector are shown: arrows, transcription start sites; SD and SA, splice donor and acceptor sites, respectively; pPGK, PGK promoters; DT-A, diptheria toxin gene; rtTA2 reverse tetracycline transactivator 2; PA, poly (A) signals.

### DETAILED DESCRIPTION OF THE INVENTION

As described herein, nuclear cloning can be employed for generating non-human mammals, particularly mice, from ES cells or cell lines without the need to first create a chimeric intermediate. The ability to derive offspring (e.g., mice) directly from ES cells or cell lines without the need to produce chimeric intermediates is a distinct advantage because it avoids the time consuming and expensive step of producing chimera and facilitates the generation of offspring with multiple genetic alterations. Applicants have demonstrated that genetic background is an important factor in cloning efficiency and a crucial parameter controlling postnatal survival of offspring that are entirely derived from ES cells. Applicants have also established that F1 ES cells are efficient donor cells for generating cloned mice. It has also been shown that these cells can be genetically manipulated prior to cloning. Because these ES cells can be maintained in culture and can be both genetically and epigenetically modified, their use in cloning makes it possible to examine which culture conditions and genes influence both pre- and post-implantation development of cloned embryos. For example, manipulating the epigenetic status of F1 ES cells in vitro prior to cloning may allow previously difficult questions, such as the collective role of imprinted genes in mammalian development, to be addressed.

The present invention relates to a method of producing a cloned non-human mammalian embryo, which can be mutant or non-mutant. The method comprises transferring a nucleus from a non-human mammalian ES cell (e.g., F1 ES cell) into an enucleated non-human mammalian oocyte, thereby producing an enucleated non-human mammalian oocyte having incorporated therein the nucleus from the non-human mammalian ES cell; activating the enucleated non-human mammalian oocyte having incorporated therein the nucleus from the non-human mammalian ES cell, thereby producing an activated oocyte; and culturing the activated oocyte under conditions appropriate for blastocyst (embryo) development, whereby a cloned non-human mammalian embryo is produced. In one embodiment, the ES cell is cultured under conditions that result in reduction of the percentage of ES cells in S phase (compared to the percentage of ES cells in S phase resulting from culturing of the ES cells in, for example, 15% fetal calf serum). In one embodiment, the ES cells are cultured in 5% fetal calf serum. In a particular embodiment, the non-human mammalian ES cell is a mouse cell and the enucleated non-human mammalian oocyte is a mouse cell. The ES cell can be a non-targeted (unmodified) F1 ES cell or a targeted ES cell, such as a targeted ES cell that comprises a gene introduced into a site in genomic DNA of the cell (e.g., by homologous recombination).

In a particular embodiment, the present invention relates to a method of producing a cloned non-human mammalian embryo, which can be mutant or non-mutant. The method comprises transferring a nucleus from a non-human mammalian F1 ES cell into an enucleated non-human mammalian oocyte, thereby producing an enucleated non-human mammalian oocyte having incorporated therein the nucleus from the non-human mammalian F1 ES cell; activating the enucleated non-human mammalian oocyte having incorporated therein the nucleus from the non-human mammalian F1 ES cell, thereby producing an activated oocyte; and culturing the activated oocyte under conditions appropriate for blastocyst (embryo) development, whereby a cloned non-human mammalian embryo is produced. In one embodiment, the F1 ES cell is cultured under conditions that result in reduction of the percentage of ES cells in S phase (compared to the percentage of ES cells in S phase resulting from culturing of the F1 ES cells in, for example, 15% fetal calf serum). In one embodiment, the F1 ES cells are cultured in 5% fetal calf serum, as described herein. In a particular embodiment, the non-human mammalian F1 ES cell is a mouse cell and the enucleated non-human mammalian oocyte is a mouse cell. In a specific embodiment, the non-human mammalian F1 ES cell is a 129SvJae×C57BL/6 ES cell. In an additional embodiment, the enucleated non-human mammalian oocyte is a B6D2F1 enucleated oocyte. In a further specific embodiment, the non-human mammalian F1 ES cell is a 129vJae×C57BL/6 ES cell and the enucleated non-human mammalian oocyte is a B6D2F1 enucleated oocyte. The F1 ES cell can be a non-targeted (unmodified) F1 ES cell or a targeted F1 ES cell, such as a targeted F1 ES cell that comprises a gene introduced into a site in genomic DNA of the cell (e.g., by homologous recombination). In one embodiment, the site in genomic DNA of the cell is a ROSA locus, such as the ROSA26 locus.

The invention also relates to a method of producing a cloned non-human mammal, which can be a mutant or non-mutant animal, such as a mutant or non-mutant mouse. As described herein, it has now been shown that mutant non-human mammals can be produced without the intermediate step of producing chimeric animals. In particular, targeted or mutant mice have been produced and the present invention is described in detail by describing their production. However, the present invention can be used to produce mutants or non-mutants of any mammal for which embryonic stem (ES) cells can be obtained.

In one embodiment, the invention is a method of producing a cloned non-human mammal. The method comprises transferring into an appropriate foster mother (e.g., a pseudopregnant female of the same species) an embryo produced by transferring a nucleus from a non-human mammalian ES cell (e.g., F1 ES cell) into an enucleated non-human mammalian oocyte of the same species, thereby producing an enucleated non-human mammalian oocyte having incorporated therein the nucleus from the non-human mammalian ES cell; activating the enucleated non-human mammalian oocyte having incorporated therein the nucleus from the non-human mammalian ES cell, thereby producing an activated oocyte; and culturing the activated oocyte under conditions appropriate for blastocyst (embryo) development, a result of which is production of a cloned non-human mammalian embryo (at least one/one or more embryo). The foster mother is maintained under conditions appropriate for production of offspring, thereby producing a non-human mammal. A particular embodiment of this invention is a method of producing a cloned mouse, comprising transferring into a pseudopregnant female mouse an embryo produced by the method described above, wherein the non-human mammalian ES cell is a mouse cell and the enucleated non-human mammalian oocyte is a mouse cell and maintaining the female mouse under conditions appropriate for production of offspring, thereby producing a cloned mouse.

A specific embodiment of this invention is a method of producing a cloned non-human mammal, comprising transferring into an appropriate foster mother (e.g., pseudopregnant female of the same species) an embryo produced by transferring a nucleus from a non-human mammalian F1 ES cell into an enucleated non-human mammalian oocyte of the same species, thereby producing an enucleated non-human mammalian oocyte having incorporated therein the nucleus from the non-human mammalian F1 ES cell; activating the enucleated non-human mammalian oocyte having incorporated therein the nucleus from the non-human mammalian F1 ES cell, thereby producing an activated oocyte; and culturing the activated oocyte under conditions appropriate for blastocyst (embryo) development, a result of which is production of a cloned non-human mammalian embryo (at least one/one or more embryo). The foster mother is maintained under conditions appropriate for production of offspring, and a non-human mammal is produced. A particular embodiment of this invention is a method of producing a cloned mouse, comprising transferring into a pseudopregnant female mouse an embryo produced by the method described above, wherein the non-human mammalian F1 ES cell is a mouse cell and the enucleated non-human mammalian oocyte is a mouse cell and maintaining the female mouse under conditions appropriate for production of offspring, thereby producing a cloned mouse. A particular embodiment of this invention is a method of producing a cloned mouse, comprising transferring an embryo produced using a non-human mammalian F1 ES cell that is a 129vJae×C57BL/6 ES cell and an enucleated mouse oocyte such as a B6D2F1 enucleated oocyte.

In another embodiment, the invention is a method of producing a cloned mutant non-human mammal. The method comprises transferring into an appropriate foster mother (e.g., pseudopregnant female of the same species) an embryo produced by transferring a nucleus from a non-human mammalian ES cell comprising at least one mutation or alteration, into an enucleated non-human mammalian oocyte of the same mammalian species, thereby producing an enucleated non-human mammalian oocyte having incorporated therein the nucleus from the non-human mammalian ES cell; activating the enucleated non-human mammalian oocyte having incorporated therein the nucleus from the non-human mammalian ES cell, thereby producing an activated oocyte; and culturing the activated oocyte under conditions appropriate for blastocyst (embryo) development, a result of which is production of a cloned mutant non-human mammalian embryo (at least one/one or more embryo). The foster mother is maintained under conditions appropriate for production of offspring, thereby producing a mutant non-human mammal. The mutations can be non-random or targeted or, alternatively, can be introduced randomly or in a non-targeted manner. A particular embodiment of this invention is a method of producing a cloned mutant mouse, comprising transferring into a pseudopregnant female mouse an embryo produced by the method described above, wherein the non-human mammalian ES cell is a mouse ES cell comprising at least one mutation or alteration and the enucleated non-human mammalian oocyte is a mouse cell and maintaining the female mouse under conditions appropriate for production of offspring, thereby producing a cloned mutant mouse.

A specific embodiment of this invention is a method of producing a cloned mutant non-human mammal, comprising transferring into an appropriate foster mother (e.g., pseudopregnant female of the same species) an embryo produced by transferring a nucleus from a non-human mammalian F1 ES cell comprising at least one mutation or alteration, into an enucleated non-human mammalian oocyte of the same species, thereby producing an enucleated non-human mammalian oocyte having incorporated therein the nucleus from the non-human mammalian F1 ES cell; activating the enucleated non-human mammalian oocyte having incorporated therein the nucleus from the non-human mammalian F1 ES cell, thereby producing an activated oocyte; and culturing the activated oocyte under conditions appropriate for blastocyst (embryo) development, a result of which is production of a cloned mutant non-human mammalian embryo (at least one/one or more embryo). The foster mother is maintained under conditions appropriate for production of offspring, and a mutant non-human mammal is produced. A particular embodiment of this invention is a method of producing a cloned mutant mouse, comprising transferring into a pseudopregnant female mouse an embryo produced by the method described above, wherein the non-human mammalian F1 ES cell is a mouse F1 ES cell comprising at least one mutation or alteration and the enucleated non-human mammalian oocyte is a mouse cell and maintaining the female mouse under conditions appropriate for production of offspring, thereby producing a cloned mutant mouse. A particular embodiment of this invention is a method of producing a cloned mutant mouse, comprising transferring an embryo produced using a non-human mammalian F1 ES cell that is a 129vJae×C57BL/6 ES cell comprising at least one mutation or alteration and an enucleated mouse oocyte such as a B6D2F1 enucleated oocyte.

Methods for activating non-human mammalian oocytes are known and readily available in the art (see, e.g., Machaty, Z. et al., Reprod. Fertil. Dev., 10(7-8):599-613 (1998); Polejaeva, I. A. et al., Nature, 407(6800):86-90 (2000); Du, F. et al., Reprod. Nutr. Dev., 35(6):703-712 (1995); Susko-Parrish, J. L. et al., Dev. Biol., 166(2):729-739 (1994); and Dominko, T. et al., Biol. Reprod., 60(6):1496-1502 (1999)). For example, oocytes can be activated with 10 mM Sr²⁺ in Ca²⁺ free media in the presence of 5 μg/ml of Cytochalasin B, as described herein.

ES cells used in the present method can contain at least one/one or more genetic alterations or mutations. Alternatively, as described above, ES cells used can be unmodified (have not been altered, after they are obtained, to contain a genetic alteration or mutation); such cells are used to produce non-mutant progeny by the method of the present invention. The genetic alterations or mutations that can be present in the ES cells used include, but are not limited to, transgenes (cDNA, genes or portions thereof), mutations (targeted or random), conditional mutations, targeted insertions of foreign genes, YAC and BAC sized transgenes, all or part of a chromosome, which may be from the same species as the embryo or another species, such as from a human. They include physical knockout of all or a part of a gene, functional knockout of a gene, introduction of a functional gene and introduction of DNA or a gene portion that changes the function/level of expression of a gene present in the ES cell (e.g., a promoter, enhancer or repressor). An important feature of the method of the present invention is that multiple genetic alterations, which will typically be consecutive genetic alterations but can also be simultaneous, can be made in the ES cells, thus circumventing the need for breeding to combine multiple alterations in one animal, as is required if presently available methods are used. Alterations can also be present in the ES cells as they are obtained from the zygote from which they are derived. As used herein, mutant ES cells encompass cells which comprise a mutation or mutations as obtained from the zygote which gave rise to the cells and cells which are mutated or altered after they are obtained from the zygote. Alterations can all be of the same type (e.g., all introduction of exogenous DNA) or of more than one type (e.g., introduction of exogenous DNA, gene knockout and conditional gene knockout). They can also be a combination of mutations present in the ES cells as derived from a zygote and mutations made after they are derived from a zygote. The alterations made in genomic DNA of ES cells can be chosen to produce a phenotype that is similar to (mimics) a condition that occurs in other species (e.g., humans) and the resulting mutant animal (e.g., mice) can, thus, serve as a model for that condition.

A variety of methods known to those of skill in the art can be used to alter or mutate ES cells. For example, an appropriate vector or plasmid can be used to introduce DNA into ES cells in order, for example, to integrate DNA into genomic DNA, express foreign DNA in recipient cells, cause recombination (homologous or nonhomologous) between introduced DNA and endogenous DNA or knock out endogenous gene(s), such as by means of the Cre-lox method. Alternatively, alterations or mutations can be produced by chemical methods or radiation. As described herein, gene targeting can also be used to produce mutant ES cells or cell lines.

Also the subject of this invention are cloned non-human mammals, mutants and non-mutants, such as cloned mice (mutants and non-mutants), produced by the nuclear cloning methods of the present invention; non-human embryos, mutants and non-mutants, produced by the methods of the invention; and a method of identifying a drug to be administered to treat a condition that occurs in a mammal, such as a human.

The mutant non-human mammals, such as mutant mice, can be used as a model for a condition for which a preventive or therapeutic drug is sought. A method of identifying a drug to be administered to treat a condition in a mammal comprises producing, using the method of the present invention, a mutant mouse that is a model of the condition; administering to the mutant mouse a drug, referred to as a candidate drug, to be assessed for its effectiveness in treating or preventing the condition; and assessing the ability of the drug to treat or prevent the condition. If the candidate drug reduces the extent to which the condition is present or progresses or causes the condition to reverse (partially or totally), the candidate drug is a drug to be administered to treat the condition.

The present invention is illustrated by the following Examples, which are not intended to be limiting in any way.

### EXAMPLES

### Example 1

Development of Cloned Embryos and Mice from Embryonic Stem Cells.

Embryos were produced by transfer of nuclei from ES cells (cultured for 1-5 days without feeder cells in standard ES media containing 1000 U/ml leukaemia inhibitory factor (LIF), but only 5% fetal calf serum (FCS)) into enucleated B6D2F1 oocytes (Wakayama, T. et al., Nature, 394:369-374 (1998)). The oocytes were activated by exposure to Sr⁺⁺ containing media, then cultured in vitro for 3 days before transfer into 2.5 days post-coitum (dpc) pseudopregnant Swiss females. Cesarean sections were performed at 18.5-19.5 dpc for all recipient females followed by fostering of any live pups.

In order to assess the most suitable genetic background for cloning, cloning efficiencies using F1 (129SvJae×C57BL/6) ES cell lines and 129 (129SvJae) ES cell lines as nuclear donors were compared. Nuclear transplantation resulted in blastocyst stage clones at about similar frequency for all the ES cell donors (Table 1). However, results demonstrated a clear effect of genetic background on survival. After transfer into pseudopregnant females, seven out of 34 (21%) F1 cloned blastocysts developed to term and into healthy adults. In comparison, eight of 76 (11%) of the inbred 129 donor ES cells developed to term but all died within 24 hours of birth. The viability of the F1 derived clones was also 5-70 fold greater than the viability of clones from other ES cell lines recently reported (R1, 4% and E14, 0.3%) (Wakayama, T. et al., Proc. Natl. Acad. Sci. USA, 96:14984-14989 (1999)). Thus, the F1 ES cells were much more efficient than the inbred 129 (J1 and E14) ES cells and the out-crossed 129 R1 ES cells as donors for nuclear transfer. Indeed, the ability of ES cells to generate viable cloned offspring might be correlated with their degree of polymorphism at 37 different SSLP markers—J1 (0/37), E14 (0/37), R1 (12/37) and Applicants' F1 line (28/37) (Simpson, E. M. et al., Nature Genet., 16:19-27 (1997)).

Comparison of the developmental potential of clones derived from ES cells with results of earlier studies of clones derived from B6C3F1 and B6D2F1 somatic cells also showed significant differences. Pre-implantation development of inbred 129 and F1 ES cell nuclei transferred into enucleated oocytes resulted in approximately 15% blastocysts (Table 1), which was less efficient than that reported for cumulus and tail-tip cell derived clones (50-60%). However, the post-implantation development and survival of 21% of the F1 ES cell cloned blastocysts to adulthood was improved compared with the 1.6% and 0.4% survival rate of clones derived from cumulus and tail tip donor blastocysts, respectively (Wakayama, T. et al., Nature, 394:369-374 (1998); and Wakayama, T. et al., Nature Genet., 22:127-128 (1999)).

The initial development of clones to the blastocyst stage may be dependent on the compatibility between the cell cycles of the donor nucleus and the oocyte (Campbell, K. H. et al., Rev., Reprod, 1:40-46 (1996)). Thus, the predominantly G0/G1 cumulus cells may be favored over rapidly cycling ES cells and methods to purify or arrest ES cells at specific stages of the cell cycle may facilitate the generation of blastocysts. Wakayama et al. (Wakayama, T. et al., Proc. Natl. Acad. Sci. USA, 96:14984-14989 (1999)) recently showed that ES cells in either the G1 or G2/M phase of the cell cycle could develop to term after nuclear transfer. Therefore, it may be that only S-phase cells, which comprise 35-40% of ES cells, are incompatible with cloning. In the work described herein, Applicants' ES cells were cultured in 5% rather than 15% fetal calf serum because the lower concentration mildly reduced the percentage of ES cells in S-phase and increased the percentage in G2/M, as determined by propidium iodide staining and FACS analysis. Further steps to synchronize the donor ES cells by FACS sorting or elutriation may improve cleavage stage development and cloning efficiency.

Data provided herein support a role for the epigenetic status and pluripotency of ES cells in determining post-implantation development. The fact that ES-cell clones have a higher rate of post-implantation development, compared with somatic cell derived clones might be due to the epigenetic status and pluripotency of ES cells. ES cell nuclei may require less epigenetic reprogramming than cumulus cell nuclei to enable full development. Further support for the role of epigenetics in post-implantation development is suggested by the development to term of clones from low passage 129 cells. Clones from high passage 129 cells died during midgestation (Table 1). This may be due to a progressive loss of epigenetic marks associated with imprinted genes during culture as seen by Dean et al (Dean, W. et al., Development,125:2273-2282 (1998)) for high passage ES cells. Thus, both the genetic background and epigenetic status of the F1 ES cells contribute to the highest efficiency for cloning cultured donor cells described to date.

To test whether nuclear cloning could be utilized to generate transgenic mice with targeted insertions, a tet-transactivator gene (rtTA2ΔSD) was integrated into the ROSA26 locus by homologous recombination in F1 (v6.5) ES cells. The ES cell clone, rtTA2ΔSD-18, which carried the targeted insert, was used as the donor for nuclear transplantation and produced a healthy, fertile cloned mouse carrying the insert (FIGURE). In a control experiment, two additional mice were cloned from an F1 subcloned cell line (v6.5 sc84). A different targeted line LJG-13 of 129 strain background gave 4 embryos which died at mid-gestation (Table 1).

Results are presented in Table 1. In Table 1, F1 refers to the 129SvJae×C57BL/6 genetic background. Surviving cloned animals were all derived from donor F1 ES cell nuclei because all had agouti coat color, while donor oocytes came from black, non agouti B6D2F1 females and the cloned embryos had been transferred into pseudopregnant white Swiss females. Targeted ES cell lines are: rtTA2ΔSD-18 and LGJ-13. a: ES cells were targeted or subcloned at 4-5 passage and grown for another 4 passages before transplantation. b: High passage. c: Only embryos which had reached morula or blastocyst stage were transferred.

These experiments indicate that nuclear cloning can be employed for generating mice with targeted insertions without generating chimeric mice as is necessary in conventional gene targeting techniques. Therefore, with improved efficiency, ES cell cloning may shorten the time required to generate mutant mice, such as knock-out mice.

TABLE 1

| | | | | Embryos^{c} | At Term | | |
|---|---|---|---|---|---|---|---|
| Genetic | | | Activated | transferred | | Alive | Survived |
| background | Cell line | Passage | oocytes | (% activated) | Dead | (% transferred) | |
| F1 | v 6.5 | 5-6 | 149 | 21 (14%) | 0 | 4 | 4 (19%) |
| F1 | v6.5 sc84 | 8-9^{a} | 46 | 6 (13%) | 2 | 2 | 2 (33%) |
| F1 | rtTA2ΔSD-18 | 8-9^{a} | 32 | 7 (22%) | 0 | 1 | 1 (14%) |
| Total | | | 227 | 34 (15%) | 2 | 7 (21%) | 7 (21%) |
| 129 | v 18.6 | 5-6 | 107 | 19 (19%) | 1 | 4 | 0 |
| 129 | J1 | 11 | 145 | 26 (18%) | 2 | 4 | 0 |
| 129^{(high)} | J1 | 35^{b} | 92 | 23 (25%) | 10 | 0 | 0 |
| 129 | LJG-13 | 8-9^{a} | 74 | 8 (11%) | 4 | 0 | 0 |
| Total | | | 418 | 76 (18%) | 17 | 8 (11%) | 0 (0%) |

The following materials and methods were used in the work described in Examples 2 to 4.

### Production of ES Cell Clones

Nuclear transfer of ES cell nuclei into enucleated metaphase II oocytes was carried out as described in Example 1 (see also Wakayama, T et al., Nature, 394:369-374 (1998); Wakayama, T. et al., Nat. Genet., 22:127-128 (1999); Ogura, A. et al., Biol. Reprod., 62:1579-1584 (2000); and Wakayama, T. et al., Proc. Natl. Acad. Sci. USA, 96:14984-14989 (1999)). One to three hours after nuclear transfer, oocytes were activated for 5 hours with 10 mM Sr²⁺ in Ca²⁺ free media in the presence of 5 μg/ml of Cytochalasin B. Embryos were cultured in vitro to the blastocyst stage and transferred to recipient mothers.

### Embryo Culture

All embryo culture was carried out in microdrops on standard bacterial Petri dishes (Falcon) under mineral oil (Squibb). Modified Chatot, Ziomek, Bavister (CZB) media (Chatot, C. L. et al., Biol. Reprod., 42:432-440 (1990)) was used for embryo culture unless otherwise noted. Hepes-buffered CZB (Chatot, C. L. et al., Biol. Reprod., 42:432-440 (1990)) was used for room temperature operations while long-term culture was carried out in bicarbonate-buffered CZB at 37° C. with an atmosphere of 5% CO₂ in air.

### Preparation of Two Cell Embryos for Electrofusion

B6D2F1 females are superovulated by i.p. injection of 7.5 units of pregnant mares' serum (Calbiochem) followed by 46-50 hours later with 7.5 units of human chorionic gonadotropin (HCG) (Calbiochem). After administration of HCG, females were mated with B6D2F1 males. Fertilized zygotes were isolated from the oviduct 24 hours later. Zygotes were left in Hepes-buffered CZB with 0.1% bovine testicular hyaluronidase for several minutes at room temperature to remove any remaining cumulus cells. After washing, zygotes were transferred to a new culture dish containing drops of biocarbonate-buffered CZB and placed at 37° C. overnight to obtain two-cell embryos.

### Culture of ES Cells

Derivation, culture and targeted mutagenesis of ES cells were carried out as previously described (Hogan, B. et al., Manipulating the Mouse Embryo: A Laboratory Manual, Cold Spring Harbor Laboratory Press, Plainview, N.Y., pp. 253-289 (1994)) with ES cell lines derived from both inbred and F1 blastocysts. ES cells were cultured in DMEM with 15% FCS containing 1,000 units/ml leukocyte inhibiting factor on gamma-irradiated primary feeder fibroblasts. For blastocyst injection, ES cells were trypsinized, resuspended in DMEM, and first preplated on a standard 10-cm tissue culture dish for 30 minutes to remove feeder cells and debris.

### Recipient Females and Cesarean Sections

Ten injected blastocysts were transferred to each uterine horn of 2.5 days postcoitum pseudopregnant Swiss females that had mated with vasectomized males. Recipient mothers were sacrificed at 19.5 days postcoitum and pups were quickly removed from the uterus. After cleaning fluid from their air passages, pups were placed under a warming light and respiration was observed. Surviving pups were fostered to lactating BALB/c albino mothers.

### Example 2

Nuclear Transfer with F1 and Inbred ES Cells.

Donor nuclei derived from four different ES cell lines of three inbred backgrounds (129/Sv, C57BL/6 and BALB/c) with six different F1 lines (126/Sv×C57BL/6, C57BL/6×129/Sv, BALB/c×129Sv, 129/Sv×Mus castaneus, C57BL/6 ×BALB/c and 129/Sv×FVB) were compared in nuclear cloning experiments. Cells from each of these ES cell lines can contribute efficiently to the germ line after incorporation into chimeric animals. 817 oocytes were reconstructed and activated by using inbred ES cell donor nuclei and 783 oocytes were reconstructed and activated by using F1 ES cell donor nuclei as judged by pronucleus (PN) formation. The efficiency of PN formation for all cell lines, inbred or F1, was approximately 70%. Activated oocytes with a visible PN derived from either an inbred or F1 nucleus developed to the blastocyst stage with about 20% efficiency. The efficiency of cleavage-stage development was similar for clones derived from the four inbred and five F1 ES cell lines, indicating that neither genetic background nor genetic heterozygosity influence in vitro preimplantation development of ES cell clones.

To assess full-term development of inbred and F1 ES cell clones, blastocysts were transferred to pseudopregnant recipient mothers. When delivered by caesarian section at embryonic day 19 of gestation, 15 of 182 cloned inbred blastocysts (8%) and 28 of 169 cloned F1 blastocysts (17%) were found to have developed to term. However, all inbred clones died within a few minutes after delivery of apparent respiratory failure (Table 2). In contrast, 78% of clones (22 of 28 pups) derived from the various F1 ES cell donors initiated breathing and developed into healthy adults (Table 3). As all pups derived from inbred ES cells died at birth and the majority of clones derived from F1 ES cell nuclei survived, these results demonstrate that heterozygosity of the donor cell genome is critical for the survival of ES cell clones. In addition, the results demonstrate that genetic heterozygosity is of general, rather then anecdotal, importance in the survival of mice cloned from ES cells by nuclear cloning.

TABLE 2
Survival of Inbred ES Cell Clones

| | | | Blastocyst | Pups alive | Pups |
|---|---|---|---|---|---|
| ES cell | | Total | stage- | at term | surviving to |
| line | Genotype | active PN | ET (% PN) | (% ET) | adulthood |
| J1 | 129/Sv | 352 | 68 (19) | 6 (9) | 0 |
| V18.6 | 129/Sv | 178 | 40 (22) | 7 (18) | 0 |
| V26.2 | C57BL/6 | 164 | 40 (24) | 2 (5) | 0 |
| V39.7 | BALB/c | 123 | 34 (28) | 0 (0) | 0 |
| Total | — | 817 | 182 (22) | 15 (8) | 0 |

Total active PN refers to the number and percent of reconstructed oocytes with observable PN. Blastocyst stage-ET refers to the number and percent of embryos with PN that developed to the blastocyst stage and subsequently were transferred to pseudo-pregnant recipient females.

TABLE 3
Survival of F1 ES Cell Clones

| | | | | Pups | Pups |
|---|---|---|---|---|---|
| | | Total | Blastocyst | alive | surviving |
| ES cell | | active | stage-ET | at term | to adulthood |
| line | Genotype | PN | (% PN) | (% ET) | (% alive) |
| V6.5* | C57B/6 × 129/Sv | 381 | 79 (21) | 18 (23) | 15 (80) |
| 129B6 | 129/Sv × C57BL/6 | 66 | 18 (27) | 3 (17) | 2 (67) |
| F_{1.2-3} | 129/Sv × M. cast. | 143 | 27 (18) | 3 (11) | 2 (67) |
| V8.1 | 129/Sv × FVB | 69 | 19 (28) | 2 (11) | 2 (100) |
| V17.2 | BALB/c × 129/Sv | 99 | 21 (21) | 2 (10) | 1 (50) |
| V30.30 | C57BL/6 × BALB/c | 25 | 5 (20) | 0 | 0 |
| Total | | 783 | 169 (22) | 28 (17) | 22 (78) |

Total active refers to the number and percent of reconstructed oocytes with observable PN. Blastocyst stage-ET refers to the number and percent of embryos with PN that developed to the blastcyst stage and subsequently were transferred to pseudo-pregnant recipient females.
*Includes three independent subclones targeted at the Rosa26 locus.

### Example 3

Inbred ES Cell-Derived Animals Die of Respiratory Failure.

ES cell pups and clones derived from inbred ES cells appeared to suffer from respiratory distress after delivery. Histological analysis of both F1 and inbred completely ES cell-derived neonates was carried out. Examination of the lungs from inbred clones revealed that the alveoli were not inflated, while the lungs of newborns derived from F1 ES cells were fully inflated and alveoli were expanded. In addition, interstitial bleeding was often seen in inbred ES cell-derived mice. These observations suggest that the failure to initiate breathing and/or sustain normal circulation likely contributed to postnatal death of inbred clones.

### Example 4

Embryonic and Placental Overgrowth in ES Cell-Derived Mice.

Embryonic and placental overgrowth and dysfunction have been suggested as potential causes of neonatal mortality in cloned livestock (Young, L. E. et al., Rev. Reprod., 3:155-163 (1998); Cibelli, J. B. et al., Science, 280:1256-1258 (1998); and Wells, D. N. et al., Biol. Reprod., 60:996-1005 (1999)) and mice (Wakayama, T. et al., Nature, 394:369-374 (1998); and Wakayama, T. et al., Nat. Genet., 22:127-128 (1999)). To examine the role of increased birth and placental weight in the survival of mice completely derived from ES cells, pairwise comparisons of neonatal mice cloned from ES cells, normal pups and in vitro cultured pups were performed using the Student's t test. Data from normal pups were recorded from litters with a size less than or equal to three. In vitro cultured, control animals were generated by isolating two-cell stage embryos, culturing them to the blastocyst stage and then transferring them to recipient females. Neonatal mice cloned from ES cells were found to have a mean embryo weight of 2.1 g and a mean placental weight of 0.32 g. These weights were significantly higher than those of normal pups or in vitro cultured pups (P<0.0001 for both weights). The increase in birth weight observed in ES cell clones was occasionally severe. Birth and placenta weights of normal mice were significantly lower than those of in vitro cultured pups (P<0.004).

Both extremely large and more normally sized embryos and placentas were observed in cloned conceptuses derived from both inbred and F1 ES cells. Significantly, while both large and more normal F1 ES cell clones survived postnatally, both large and more normal-sized inbred ES cell clones died. Although it has been previously suggested that neonatal and placental overgrowth might be related to neonatal lethality in cloned animals (Young, L. E. et al., Rev. Reprod., 3:155-163 (1998); Cibelli, J. B. et al., Science, 280:1256-1258 (1998); Wells, D. N. et al., Biol. Reprod., 60:996-1005 (1999); Wakayama, T. et al., Nature, 394:369-374 (1998); and Wakayama, T. et al., Nat. Genet., 22:127-128 (1999)), the results described herein indicate no apparent correlation between placental or embryonic overgrowth and neonatal survival. The results described herein also demonstrate that either in vitro culture or transfer of embryos to pseudopregnant recipient mothers can cause increased placental and embryonic birth weight.

The teachings of all the articles and patent documents cited herein are incorporated by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of producing a cloned non-human mammalian embryo, comprising:
a) transferring a nucleus from a non-human mammalian F1 ES cell into an enucleated non-human mammalian oocyte, thereby producing an enucleated non-human mammalian oocyte having incorporated therein the nucleus from the non-human mammalian F1 ES cell;
b) activating the enucleated non-human mammalian oocyte having incorporated therein the nucleus from the non-human mammalian F1 ES cell, thereby producing an activated oocyte; and
c) culturing the activated oocyte under conditions appropriate for blastocyst development,
whereby a cloned non-human mammalian embryo is produced.

2. The method of claim 1, wherein the non-human mammalian F1 ES cell is a mouse cell and the enucleated non-human mammalian oocyte is a mouse cell.

3. The method of claim 2, wherein the non-human mammalian F1 ES cell is a 129SvJae×C57BL/6 ES cell.

4. The method of claim 3, wherein the enucleated non-human mammalian oocyte is a B6D2F1 enucleated oocyte.

5. The method of claim 2, wherein the F1 ES cell is a targeted F1 ES cell.

6. The method of claim 5, wherein the targeted F1 ES cell comprises a gene introduced into a site in genomic DNA of the cell.

7. The method of claim 6, wherein the gene introduced into the site in genomic cell is introduced by homologous recombination.

8. The method of claim 7, wherein the site in genomic DNA of the cell is the ROSA26 locus.

9. A cloned non-human mammalian embryo produced by the method of claim 1.

10. A cloned non-human mammalian embryo produced by the method of claim 2, wherein the cloned embryo is a cloned mouse embryo.

11. A cloned non-human mammalian embryo produced by the method of claim 3.

12. A cloned non-human mammalian embryo produced by the method of claim 4.

13. A cloned non-human mammalian embryo produced by the method of claim 5.

14. A cloned non-human mammalian embryo produced by the method of claim 6.

15. A cloned non-human mammalian embryo produced by the method of claim 7.

16. A cloned non-human mammalian embryo produced by the method of claim 8.

17. A method of producing a cloned non-human mammal, comprising transferring an embryo produced by the method of claim 1 into an appropriate foster mother and maintaining the foster mother under conditions appropriate for production of offspring, thereby producing a non-human mammal.

18. A method of producing a cloned mouse, comprising transferring an embryo produced by the method of claim 2 into a pseudopregnant female mouse and maintaining the female mouse under conditions appropriate for production of offspring, thereby producing a cloned mouse.

19. A method of producing a cloned mouse, comprising transferring an embryo produced by the method of claim 3 into a pseudopregnant female mouse and maintaining the female mouse under conditions appropriate for production of offspring, thereby producing a cloned mouse.

20. A method of producing a cloned mouse, comprising transferring an embryo produced by the method of claim 4 into a pseudopregnant female mouse and maintaining the female mouse under conditions appropriate for production of offspring, thereby producing a cloned mouse.

21. A method of producing a cloned mouse, comprising transferring an embryo produced by the method of claim 5 into a pseudopregnant female mouse and maintaining the female mouse under conditions appropriate for production of offspring, thereby producing a cloned mouse.

22. A cloned mouse produced by the method of claim 18.

23. A cloned mouse produced by the method of claim 19.

24. A cloned mouse produced by the method of claim 20.

25. A cloned mouse produced by the method of claim 21.
